(19) United States
(12) Patent Application Publication
(10) Pub. No.: US 2012/0137930 A1
(43) Pub. Date: Jun. 7, 2012

(54) Coating Compositions And Process For The Preparation Thereof

(75) Inventors: Erik Alexander Bijpost, Nieuwegein (NL); Alexander Maslow, Deventer (NL); Jacobus Gerardus Korver, Wormer (NL)

(73) Assignee: Holland Novochem B.V., Nieuwegein (NL)

(21) Appl. No.: 12/996,076

(22) PCT Filed: Jun. 15, 2009
(86) PCT No.: PCT/NL2009/000133

§ 371 Date: Feb. 24, 2012

(30) Foreign Application Priority Data
Jun. 13, 2008 (NL) 1035576

(51) Int. Cl. C08K 5/09; B01J 2/30; C11C 1/02; B05D 7/24; C07C 229/02; C07C 59/00

(52) U.S. Cl. 106/504; 554/103; 554/112; 554/110; 554/121; 554/213; 554/161; 427/212

(57) ABSTRACT

A composition and processes for the reduction of the tendency to baking, moisture absorption and/or dust formation of a granulate being susceptible therefore, such as fertilizers, minerals, ores, as well as to obtain stable coloring dispersions, density control of cokes and the reduction of foam in industrial processes, wherein synthetic polymers of fatty alkyl compounds, especially fatty acid esters and fatty-alcohols, fatty amines, fatty amides and fatty ethers, or distillation residues of the original monomers themselves, can be used, are disclosed. The distillation residues are preferably derived from the bottom fraction, obtained by distillation of the fraction being suitable as biodiesel during the biodiesel production process.

In the last decade it became a unmistakable clear that the amount of garbage is still growing by increasing consumption and production. On the other hand, the availability of petroleum is final. It is therefore especially necessary to investigate where garbage can be converted in valuable products, by which on the one side the use of petroleum is restricted and on the other side new alternatives can be developed and prove itself. This problem is applied to many fields of industry, varying from motor fuel to industrial applications.

So, world-wide many solid compounds, such as inorganic salts, are produced and marketed. Salts are among others used as a fertilizer and drying agent or as an additive for food, cleaning agents and the production of glass and porcelain.

Before it reaches its final use, inorganic salts often undergo a long term storage and/or intercontinental transport. Therefore the salt granulate may not stick together (also called baking), may not absorb moisture and/or produce dust. To obtain solid granulates, small amounts of chemicals are added to the salts. A known example is for example potash for sodium chloride (see for example U.S. Pat. No. 3,174,825).

In the field of fertilizers, almost always use is made of a fatty amine (C12-C22), especially tallow fatty amine (C16-C18), which is dissolved in a mixture of oil and paraffins. These compositions can, possibly in combination with talc, prevent caking of salts (see for example U.S. Pat. No. 4,150,965) and after addition of alkyl phosphate esters also strongly reduce the moisture absorption (see for example EP 0 113 687). These mixtures are mostly brought onto the fertilizer by means of a coating drum or by means of a spraying system at an endless conveyer. By that a thin film layer is formed on the granulate by which it is (partially) protected against influences from the outside. Coatings on the basis of oil and paraffins are studied very intensively and several compositions have been patented (see for example WO 03/006399 and the references therein). The power is that these coatings usually give an excellent performance at very low dosages. Commonly used amounts are between 0.05 and 0.15 percent by weight based on the weight of the treated fertilizer.

The legislation in Europe on the field of the treatment, of salts, especially fertilizers, is becoming more and more severe. It means that there is a need to environmentally friendly products, which give salts such a protection that they can be stored and processed without problems.

Apart from that is the demand to petroleum and derived products, such as mineral oil and paraffins, very strongly increased. It means that the availability decreases and the prices increase strongly. Moreover the amount of waste is becoming larger worldwide by the increasing consumption and production. It is very desirable to convert as much as possible waste in valuable products.

For the industry of fertilizers there is a strongly felt need to alternative coatings which suffice the international legislation, are good available and result in a price advantage.

Caking of ammonium nitrate containing fertilizers can be reduced by using poly alkylene glycols as a coating (GB 1,026,023). Most poly alkylene glycols are soluble in water, low-toxic and biologically decomposable. The optimum dosage is between 0.3 and 1.0 percent by weight based on the weight of the treated fertilizer. This is considerably higher than usual with oil-paraffin containing products. Poly alkylene glycols are considerably more expensive than the present coating products.

The use of alkyl amine-alkyl naphthalene sulfonate mixtures (U.S. Pat. No. 4,374,039) and alkyl sulphates (U.S. Pat. No. 4,772,308) as an antibaking coating for salts and fertilizer is also disclosed. Practice has shown that these products can give a maximum reduction, of lump formation of 50%. Moreover fertilizers, which have been treated with sulphonates or sulphates show, after long term storage cake and dust formation. By moisture absorption of the fertilizer, strong surface active alkyl naphthalene sulphonates or alkyl sulphates can dissolve a part of the fertilizer, by which the fertilizer can easily coagulate and form dust particles.

The baking tendency of sodium chloride can be strongly reduced by treating it with metal complexes of hydroxy poly carboxylic acids, preferably an iron complex of meso-tartaric acid (WO 00/59828). It appears that these compounds do not suffice as treating agent for salts with a strong tendency to lump formation and/or moisture absorption, such as most kinds of fertilizers, for example NPK's and ammonium nitrates. Another approach has been reported in WO 01/38263 and WO 02/090295. Therein, a part of the paraffin and oil has been replaced by natural oils, such as rapeseed oil. The products are inherently biologically decomposable. The natural oils are nevertheless very sensitive to polymerization and oxidation, by which it is only restricted perishable, especially at higher storage temperatures. In practice it has therefore been shown that such compositions can't be processed as a coating, because it results in the premature blockage of pipes and even to completely blocked tanks. Because the polymerisation and oxidation products of natural oil are badly soluble in common solvents, the owner had nothing else to do than to replace the tank: a costly operation!

Applicant has shown in an earlier research (WO 2006/091076) that natural residue products, such as corn steep liquor, combined or not with polyalkenylamines, can act excellently as an anti-caking agent for several fertilizers. It has been shown that aqueous products are not desired for all kinds of fertilizers. For some NPK's the addition of water can result in an increase of coagulation.

Some fertilizers, especially nitrate containing salts, are extremely sensible for the absorption of water. To reduce the baking tendency stabilizers, such as magnesium nitrate or aluminium sulfate, are often added. Because of this, the critical relative air humidity of the end product becomes even lower. Such kinds of fertilizers must be treated with a moisture proof coating on the basis of (linear) paraffin wax (see WO 03/006399).

Due to improved refining processes and increasing demand, the market supply of linear paraffins is strongly reduced. It is very desirable to have a comparable or better alternative.

For several uses in the fertilizer industry, especially the consumer market, it is desired to provide the granulate with a color. This coloring agent is dissolved in a suitable solvent or dispersed in a low-viscosity mineral oil. Water soluble coloring agents are less suitable for coloring fertilizers, because they can be extremely sensible for color changes at variation in pH. Pigments are on the contrary processed in fertilizer coatings. It is of great importance that the coloring pigment in the mixture does not coagulate and maintains a low viscosity. Low-viscosity mineral oil is more and more scarce and expensive due to the use as a lubricant for engines. Therefore there is a demand and need to an alternative solvent or carrier for coloring agents and pigments. This is not restricted only to the coloring of fertilizers, but is also applicable to uses as the coloring of concrete, asphalt or even wood shavings.

The restricted availability and increased price of mineral oil also rises problems in the desired pretreatment of cokes. Before it is processed in the cokes oven, it must be treated with an apolar oil to control the bulk density such that large pressure differences will not result in the oven. For the time being, the cokes is treated with mineral oil. Recently a good action has been described to monomeric residues from the biodiesel production process (WO 2009/039070). Due to the relatively high polarity of the raw materials, such as glycerin, it indeed deviates strongly from the conventional mineral oil. In the cokes industry there is a need to products which strongly resemble the common mineral oil, are good available and have a low cost price.

Another very common problem in industrial processes is the production of foam during production and/or cleaning processes. This can be reduced by the addition of defoamers. Known products are organosilicon compounds, fatty acid esters and mineral oil. Organosilicon containing compounds are mostly very effective but expensive. The defoaming action of fatty acid esters and mineral oil is less, whereas they are more restricted in stock in recent years. In other words, there is a need to an alternative.

### Aim of the Invention

Due to the increasing amount of waste products there is a need to convert them in useful substances. With that a surplus value is created for mankind and environment. During the processing of natural oils to for example biodiesel, fatty alcohols, stearic acid or tallow fatty amine, all kinds of residue flows are released in bulk amounts. They are mostly burned or stored as being waste.

An aim of the invention is thus the provision of a use of waste products from the natural oil processing industry as a valuable end product.

Due to the large increase in prices and availability of mineral oil and paraffins and the legislation which is becoming more stringent with respect to the treatment of salts, especially plant auxiliary substances, such as fertilizers, there will be an increasing demand to a product, which can be used instead of these known products as an additive to reduce the tendency to caking, the moisture absorption of the grains and the dust formation. This product must meet the following conditions:
a good performance
being added in a small amount at preferably the surface of the grains
being cheap
being mineral oil free
being long perishable at high storage temperatures (80-90° C.)
being environmentally friendly

The aim of the invention is thus to provide a composition for the preparation of a mineral oilfree, freeflowing granulate of a plant auxiliary substance.

It has now surprisingly been found by Applicant that a certain group of polymers is extremely suitable as a coating composition for granulate particles which are sensitive to caking, moisture absorption, and formation of dust particles, in a degree which is acceptable for the use of those granulate particles.

Said coating composition consists more especially of a polymer compound having formula

((FA)ₚ−X))_{q} (I)

wherein

FA is a saturated or non-saturated, C₈-C₂₄ fatty alkyl, having 0 to 5, preferably from 0 to 3, C═C groups in the chain,

X is a residue which is selected from —C(O)OH, —C(O)OR, —NH₂, —NHC(O)R′, NHC(O)OH, —OH, —COR

p=1 to 1000, preferably from 1 to 3,

q=1 to 1000, preferably from 2 to 5,

provided that p and q are not both equal to 1 at the same time,

R is a lower alkylgroup,

R′ is H or a lower alkyl group

Such polymer compounds are based on unsaturated monomers containing fatty alkyl groups. Depending upon the site of the unsaturation in the fatty alkyl chain, the polymerized product has a straight or branched chain, and can be a di-, tri-, oligo-, or polymer. For the sake of simplicity, there is always talk of a polymer here.

According to an attractive embodiment, R and R′, which can be the same or different, are equal to CH₃, C₂H₅, C₃H₇, C₄H_{9 C}₅H₁₁, pentaerytritol residue, glycerol residue, or an ether bond.

More preferably, the group FA is a saturated or unsaturated, C₁₄-C₂₂ fatty alkyl, having from 0 to 5, preferably 0 to 3, C═C groups in the chain.

Another aim of the invention is the advantageous use of distillation residues, especially polymers (polymers concern oligo- and polymers) from the production of biodiesel and related products, such as fatty acids, fatty alcohols, fatty amines, fatty amides, and fatty ethers.

It has been found by Applicant that these distillation residues contain polymer compounds having the above-mentioned formula (I) and can therefore excellently act as additives for plant auxiliary substances, such as fertilizers, and by which the tendency to caking, moisture absorption and/or dust formation can be reduced to a minimum.

The coating composition according to the invention is thus preferably derived from the bottom fraction of the biodiesel distillation process.

The polymer compound according to the invention is nevertheless, according to an attractive embodiment, also obtainable in a synthetic way, more especially by distillation of an ester or salt of a corresponding (monomeric) fatty acid, fatty alcohol, fatty amine, fatty amide, or fatty carbamate having 8-24 C-atoms.

Another synthesis method comprises that the polymer is polymerized from the corresponding monomer, preferably by radical initiated polymerization.

The granulate, onto which the composition according to the invention is preferably applied, is a fertilizer, more preferably an ammonium containing fertilizer.

The invention also relates to a process for the reduction of the tendency to baking, the moisture absorption and/or the dust formation of a granulate being susceptible therefore, selected from the group of a plant auxiliary substance, fertilizers, minerals and ores, in that the surface of the granulate is at least partially coated with a coating composition as disclosed above.

Before the application of the coating composition on the granulate, the polymer compound can be taken in a carrier which is suitable for that purpose, for example water, or another suitable liquid, such as an alkanol.

To improve the anti-baking properties of the granulate, further an organoamine having the general formula R″NH₂, wherein R″ is a straight or branched, cyclic or aromatic, C₁-C₃₀ alkyl or alkenyl group, can further be applied. This can be effected simultaneous, before or after the application of the polymer according to the invention.

Expediently, R″ is a straight or branched, C₁₂-C₃₀ alkyl or alkenyl group.

Further it is recommended to apply at least a compound, selected from the group of straight or branched, C₁₆-C₂₄ alkane acids; straight or branched C₁₆-C₂₄ alkoxylates; phosphate acid mono and dialkylesters having 1-24 C-atoms; or straight or branched, C₈-C₂₄ alcohols, on the granulate.

It has further been found that the presence of a polymerisation products of a 1,4 unsaturated, C₄-C₁₂ carboxylic acid or carboxylic acid anhydride onto the granulate results in an end product having excellent flowing properties.

Preferably, the unsaturated carboxylic acid or carboxylic acid anhydride, maleic acid, maleic acid anhydride, itaconic acid, or citraconic acid.

Further, preferably paraffin or petrolatum is applied onto the granulate.

Apart from, or instead of, the above-mentioned compounds, according to an embodiment of the invention, apart from at least a polymer compound according to formula (I) a surface-active compound can be applied on the granulate. This surface-active compound is preferably an organosilicon-containing compound. Such compounds are commonly known to the expert.

Another aim of the invention is the use of polymers according to formula (I), especially distillation residues from the production of biodiesel and related products, such as fatty acids, fatty alcohols, fatty amines, fatty amides, fatty ethers, as a solvent or co-solvent for dissolution and/or dispersion of coloring agents and coloring pigments.

Another aim of the invention is the use of polymers according to formula (I), especially distillation residues from the production of biodiesel and related products, such as fatty acids, fatty alcohols, fatty amines, fatty amides, fatty ethers, as a density controlling additive for the treatment of cokes.

Another aim of the invention is the use of polymers according to formula (I), especially distillation residues from the production of biodiesel and related products, such as fatty acids, fatty alcohols, fatty amines, fatty amides, as a defoamer in industrial processes.

The diverse, above-mentioned, compounds are preferably applied on the granulate particles in a dosage, of each thereof, of from 100-10.000 ppm, more preferably from 500-3.000 ppm, based on the weight of the granulate particles.

The invention further relates to a process for the preparation of the polymer compounds, as disclosed above, wherein a residue flow, which contains vegetable oils, animal fat, or recycled fat, is subjected according to a process known for the production of biodiesel, to transesterification, the thus obtained product is distilled to remove a fraction being suitable as biodiesel, and the thus obtained remaining bottom fraction is isolated to obtain a fraction containing polymer compounds having formula (I), which compounds are, if desired, subjected to functionalization to amine, amide, or carbamate compounds.

According to a very attractive embodiment, as said residue flow, a residue flow being known as yellow grease is used.

Yellow grease consists especially of old or used domestic fat, such as frying fat, of animal or vegetable origin. Any source being suitable for the production of biodiesel can of course be used.

### DESCRIPTION OF THE INVENTION

### Fertilizer

The invention relates to a process for the preparation of mineral oilfree, free-flowing granulate particles of a plant auxiliary substance, by modifying at least partially the surface of the granulate particles by treating the grains at a temperature in the range of from 0-90° C. with an additive on the basis of polymer residues, especially polymers, derived from the distillation processes of biodiesel (methyl and/or ethyl esters of natural fatty acids) and related products, such as glycerol esters (mono-, di- and triesters) and pentaerythritolesters (mono-, di-, tri- and tetraesters) as well as residues from distillation processes of fatty acids, fatty alcohols, fatty amines and fatty amides. These polymers can also be obtained through a synthetic route, preferably through radical polymerization, from the corresponding monomers.

It is observed that the treatment according to the invention can be applied to crystals, granules or prills; for the sake of simplicity, the invention is explained by means of granules.

The anti-baking action on fertilizers through coatings on the basis of fatty polymer residues can be obtained by mixing the common additives, such as alkylamines (C16-C24), alkane acids (C16-C24), alkoxylates (C16-C24), phosphate acid mono and dialkylesters (C1-C24), alkyl (C1-C24) aromatic sulphonates and combinations of these compounds. If desired, physical properties, such as sticking tendency, solidification behavior and viscosity, can be optimized by addition of paraffins or petrolatum.

Apart from that, Applicant noticed surprisingly that the addition of polymers on the basis of maleic acid anhydride, citraconic acid and/or itaconic acid to a polymer distillation residue of natural oil results in a product which as such can excellently act as a fertilizer coating.

To reduce the moisture absorption by the granulate, if desired, a biologically decomposable ethoxylated organic component can be added, which has a HLB-value between 2 and 11.

Applicant also noticed surprisingly that distillation residues of fatty alcohols (C8-C24) as such provide an excellent protection against moisture absorption to a granulate being susceptible for moisture, especially to stabilized nitrates.

### (Co)Solvent for Coloring Substances and Coloring Pigments

Moreover it appeared that polymers according to formula (I), especially distillation residues from the production of biodiesel and related products, such as fatty acids, fatty alcohols, fatty amines, fatty amides, can act excellently as a solvent or cosolvent for dissolving and/or dispersion of coloring substances and coloring pigments. The obtained product can on or in any substrate, especially fertilizers, be used to provide any desired color.

### Density Controlling Additive for Cokes

Apart from that, applicant noticed that the density of cokes can be raised by the addition of polymers according to formula (I), especially distillation residues from the production of biodiesel and related products, such as fatty acids, fatty alcohols, fatty amines, fatty amides.

### Defoamer

Applicant also found that polymers according to formula (I), especially distillation residues from the production of biodiesel and related products, such as fatty acids, fatty alcohols, fatty amines, fatty amides, can act excellently as defoamers for industrial processes, especially cleaning processes. If desired, organosilicon containing compounds can be admixed to optimize the defoaming action.

### BACKGROUND

In recent years, worldwide an enormous amount of products based on natural oil, among others derived from palm trees, algae, sunflowers and rapeseed, is produced. The best known example is biodiesel. This fuel additive is produced by esterification of natural oil with methanol or sometimes ethanol. Also several other kinds of esters are produced for other uses, such as the preparation of surface-active compounds. Because in the system several equilibrium reactions take place, among others reactions with water, esters can never be obtained completely pure in one step. Therefore these products are purified, especially by means of distillation. During the distillation of biodiesel (and related products), an enormous amount of residue remains. In the case of biodiesel, the polymer residue, consists of glycerol esters (mono, di and tri), cross-linked esters and dimeric esters up to high molecular polymer (Mw <1.000.000) products. The polymer is very good available, but is not suitable as a fuel because of the presence of high molecular fractions: the solidification point of these compounds is too high to be used as a fuel. The polymers are liquid because they are derived from unsaturated fats. During the distillation, conjugated systems are formed. This has as a practical advantage that such a product can easily be traced by means of UV and/or fluorescence. When use is made of hardened fats (read: hydrogenated), the melting point is considerably higher, depending of the degree of saturation. The polymer residues from the biodiesel distillation (and related products, such as fatty acids, fatty alcohols, fatty amines and fatty amides) have a constant quality with respect to viscosity and color. Therefore these products are interesting for several uses, for example for binding dust during coal transshipment or from other solid compounds.

### Fertilizer

Said dust binding properties of biodiesel residues have been claimed after the priority date of the present invention in patent WO 2008/100921. It is suggested that oily monomeric biodiesel residues, such as fatty acids, fatty acid esters, glycerin and glycerin esters, as such also provide sufficient action against baking of fertilizers. The kind of fertilizer has nevertheless not been mentioned. It is nevertheless known to the expert that the performance of these kinds of coating oil will be insufficient for the prevention of caking of fertilizers which are typically sensitive to baking. They are simply too hygroscopic therefore. The degree of baking of fertilizers is strongly determined by the present amount of water/moisture: the higher the moisture content, the higher the baking. Moreover a (monomeric) oily biodiesel residue will not as such be able, due to the small sticking properties, to protect dusty kinds of fertilizers (and other dusty substrates) sufficiently.

Applicant has shown that the above disadvantages can be removed by using polymers according to formula (I), especially polymeric biodiesel residues and similar fractions, derived from fatty acids, fatty alcohols, fatty amines, and fatty amides, if desired completed with common additives, especially tallow fatty amine and fatty acids, and/or admixture with paraffins and/or petrolatum.

Applicant has also shown that the polymers can be prepared by polymerization of the corresponding monomers. Preferably, the polymerization is initiated by radicals, especially organoperoxides.

Polymers on the basis of 1,4-unsaturated (mono or di) carboxylic acids or anhydrides, such as itaconic acid, maleic acid, citraconic acid and maleic acid anhydride, are for example used as a rubber additive or antiscalant. These products have not been disclosed before as an auxiliary substance to reduce the baking tendency, moisture absorption and/or dust formation of solid substances, especially fertilizers.

The advantages of the use of coatings on the basis of polymeric natural oil residues are the good availability, the favorable prize and the good environmental profile. It can act for several uses as a full alternative for mineral oil and possibly for paraffin.

### (Co)Solvent for Coloring Substances and Coloring Pigments

Coloring substances and coloring pigments for industrial-uses are often processed to a solution and a pigment slurry or paste respectively. The pigments are dispersed in a carrier liquid, for example mineral oil, and kept stable by a dispersant.

Applicant has noticed that polymers according to formula (I), especially polymeric distillation residues derived from the production of biodiesel and related products, such as fatty acids, fatty alcohols, fatty amines, fatty amides, can act good as a basis liquid for coloring pigments. The suspensions remain stable and the pigments will not become lumpy. The advantage of the use of distillation residues is that it is no longer necessary to use mineral oil.

### Density Controlling Additive for Cokes

Cokes is used in a large scale as, among others, a reduction agent for raw iron. During the processing of cokes in cokes furnaces, the wall pressure must be at an optimum. This parameter is strongly influenced by the density of the cokes. Due to the presence of vapor on the rough cokes, the density sometimes strongly varies. To control this, diesel or mineral oil is added to the cokes. It concerns very large amounts of valuable fuel/raw material. WO 2009/039070 describes-methods and compositions on the basis of monomeric biodiesel side products, such as fatty acids and glycerin.

Applicant has shown that polymers according to formula (I), especially polymeric distillation residues derived from the production of biodiesel and related products, such as fatty acids, fatty alcohol, fatty amines and fatty amides, can be a good alternative for mineral oil.

### Defoamer

Foaming is a very common problem in industrial processes, varying from the digestion of phosphate rock to the cleaning of vegetables. There are several products which can reduce foaming. The most common agent is mineral oil, mixed or not with specific surface active compounds. Such defoamers are used in a large scale and are often very expensive.

Applicant has noticed that polymers according to formula (I), especially polymeric distillation residues derived from the production of biodiesel and related products of natural origin give as such a good defoaming effect. The action can, if desired, be improved by admixing organo silicium containing compounds.

### EXPERIMENTAL

The inventions will be explained below by means of several non-restricting examples, which were executed as follows.

### Fertilizer

For applying the coating, the salt granulate (1.0 kilogram per sample) is kept in sealed plastic jars in an oven at 35° C. for 16 hours. The treatment takes place as follows: the salt is brought into a rotating coating drum, which has a temperature of at least 50° C. Thereafter, 0-10,000 ppm of a coating (T=approximately 80° C.) is sprayed or dripped onto the salt granulate, followed by rotating the drum for yet another 5 minutes. The thus treated grains are transferred into a plastic jar. After sealing the jar, the grains are allowed to cool to room temperature over a period of 24 hours.

### Process for Determining the Baking Tendency

A representative test for simulating the caking of salts was performed as follows. Polyethylene tube foil was closed on one side by means of sealing (70 mm wide, 300 mm long). On the bottom of the performed pouch, a plastic chip (48 mm in diameter) was brought. The pouch was filled with 125 grams of salt. Subsequently, another plastic chip was put onto the salt. The pouch was evacuated and closed by a seal. The obtained pouch was suspended from a metal pin. This procedure was repeated twice for the same sample. All sealed pouches with salt were subsequently placed in an autoclave. The samples were kept for a week at 0.2 bar gauge pressure at a temperature of 35° C. Subsequently the pouches were carefully cut open. The hardness of the baked sample was measured in a breaking apparatus. The force required to break the sample was read electronically. The obtained values (expressed in kilograms) were the average of at least three samples.

### Process for Determining the Moisture Absorption

Two plastic beakers of 200 ml were filled with 160 grams (blank or treated) of fertilizer. The weights of the beakers as well as the fertilizer are measured with an analytical balance. The total weight is calculated (mₜₒₜ). The beakers are placed in a climate chamber with the following conditions: 80% relative air humidity and a temperature of 20° C. After 72 hours, the samples are removed from this climate chamber and subsequently the weight is determined (mₙₐ) and the appearance is examined. The moisture absorption can thereafter be calculated as follows:

Moisture absorption=mₙₐ−mₜₒₜ

### Process for Determining the Dust Tendency

The relative amount of dust particles of the granules treated according to the invention, is studied as follows. 50 grams of granules were weight accurately and transferred into a three-neck round-bottom flask of 500 ml. The flask was provided with a glass column and a gas capillary, connected with a compressed air cylinder. The end of the gas capillary was placed in the granulate. Air (2 bar gauge pressure) was blown through the granulate for 15 seconds, by which a fluidized bed system was created. The weight of the remaining granulate was determined accurately on an analytical balance. The loss in weight is a measure for the amount of free dust particles. The granulate was recycled in the three-neck flask. For 1 minute, compressed air was blown through the granulate, by which the formation of friction dust became possible. The remaining granulate was weight on an analytical balance. The second loss in weight is a measure for the friction dust. The total amount of dust was calculated by adding both numbers and dividing by the originally weight amount of granulate, expressed in ppm. The determinations were performed in duplicate.

### Example 1

Three polymeric fatty acid methyl ester residues, whereof one contains more than 50% of polymer, were further examined. The composition is disclosed in Table 1. They are mixed with 5% (w/w) of tallow fatty amine, 4% (w/w) of tallow fatty amine/1% (w/w) of stearic acid, 4% (w/w) of tallow fatty amine/1% (w/w) of magnesium stearate. The mixtures were brought as a coating onto Calcium Ammonium Nitrate (CAN, 27% N). As a reference, a common product on the basis of 95% (w/w) of mineral oil and 5% (w/w) of tallow fatty amine was used. The baking tendency and dust tendency of the dust coated material were determined. The obtained results are presented in Tables 2 and 3.

TABLE 1

| Fatty acid ester residue | 1 | 2 | 3 |
|---|---|---|---|
| Copolymerised triglyceride (% w/w) | 30 | 40 | 70 |
| Unsaponifiable (% w/w) | 50 | 35 | 15 |
| Long chain methylester (% w/w) | 20 | 25 | 15 |

TABLE 2

| | | Baking | Dust |
|---|---|---|---|
| | Dosage | tendency | formation |
| Coating | (ppm) | (35° C.) (kg) | (ppm) |
| Blank (without a coating) | — | 15 | 150 |
| Mineral oil (visc. 80 | 900 | 14 | 60 |
| mPa · s, 40° C.) | | | |
| Reference (95% (w/w) of | 820 | 0.8 | 70 |
| mineral oil and 5% (w/w) of | | | |
| tallow fatty amine) | | | |
| Fatty acid ester residue 1 | 850 | 12 | 50 |
| Fatty ester residue 1 plus | 850 | 10 | 20 |
| 10% (w/w) petrolatum | | | |
| Fatty acid ester residue 1 | 860 | 0.9 | 55 |
| plus 5% (w/w) of tallow | | | |
| fatty amine | | | |
| Fatty acid ester residue 1 | 840 | 0.4 | 30 |
| plus 4% (w/w) tallow fatty | | | |
| amine/1% (w/w) stearic acid | | | |
| Fatty acid ester residue 1 | 850 | 0.7 | 80 |
| plus 4% (w/w) of tallow | | | |
| fatty amine/1% (w/w) | | | |
| magnesium stearate | | | |
| Fatty acid ester residue 2 | 820 | 10 | 70 |
| Fatty acid ester residue 2 | 860 | 0.8 | 65 |
| plus 5% (w/w) of tallow | | | |
| fatty amine | | | |
| Fatty acid ester residue 2 | 880 | 0.4 | 20 |
| plus 4% (w/w) of tallow | | | |
| fatty amine/1% (w/w) of | | | |
| stearic acid | | | |
| Fatty acid ester residue 2 | 850 | 0.8 | 35 |
| plus 4% (w/w) of tallow | | | |
| fatty amine/1% (w/w) of | | | |
| magnesium stearate | | | |

TABLE 3

| | | Baking | Dust |
|---|---|---|---|
| | Dosage | tendency | formation |
| Coating | (ppm) | (35° C.) (kg) | (ppm) |
| Blank (without a coating) | — | 15 | 170 |
| Mineral oil (visc. 80 | 920 | 12 | 60 |
| mPa · s, 40° C.) | | | |
| Reference (95% (w/w) of | 880 | 0.7 | 75 |
| mineral oil and 5% (w/w) of | | | |
| tallow fatty amine) | | | |
| Fatty acid ester residue 3 | 900 | 7.0 | 25 |
| Fatty acid ester residue 3 | 880 | 0.5 | 25 |
| plus 5% (w/w) of tallow | | | |
| fatty amine | | | |
| Fatty acid ester residue 3 | 900 | 0.2 | 10 |
| plus 4% (w/w) of tallow | | | |
| fatty amine/1% (w/w) of | | | |
| stearic acid | | | |
| Fatty acid ester residue 3 | 850 | 0.2 | 15 |
| plus 4% (w/w) of tallow | | | |
| fatty amine/1% (w/w) of | | | |
| magnesium stearate | | | |

The above example shows that polymer fatty acid ester residues, if provided with a common anti-baking additive, can reduce the baking tendency and dust tendency of a salt granulate, such as CAN, considerably. In this experiment it appears that polymer fatty acid ester residues are valuable alternatives for mineral oil. Besides, it has been shown that the binding of dust can further be optimized by the addition of a common paraffin, such as petrolatum.

### Example 2

Several polymeric distillation residues from different natural oil derivatives have been further examined with respect to anti-baking and dust binding capacities.

Also a pure polymer of oleic acid has been tested. The polymer has been prepared by mixing 98% (w/w) of oleic acid with 2% (w/w) of dicumene peroxide and thereafter heating it for 10 minutes at 160-190° C. After cooling, a viscous oil (52 cSt, 100° C.) results. This was used for baking and dust formation testing.

The polymers were tested in pure form and as a mixture with 5% (w/w) of tallow fatty amine. The mixtures were coated on Calcium Ammonium Nitrate (CAN, 27% N). As a reference, a common product on the basis of 95% (w/w) of mineral oil and 5% (w/w) of tallow fatty amine has been used. The results are presented in Table 4.

TABLE 4

| | | Baking | Dust |
|---|---|---|---|
| | Dosage | tendency | formation |
| Coating | (ppm) | (35° C.) (kg) | (ppm) |
| Blank (without a coating) | — | 15 | 250 |
| Mineral oil (visc. 80 | 1000 | 14 | 85 |
| mPa · s, 40° C.) | | | |
| Reference (95% (w/w) of | 950 | 0.9 | 90 |
| mineral oil and 5% (w/w) of | | | |
| tallow fatty amine) | | | |
| Fatty acid ethyl ester residue | 1000 | 6.0 | 80 |
| Fatty acid ethyl ester | 980 | 0.8 | 75 |
| residue plus 5% (w/w) of | | | |
| tallow fatty amine | | | |
| Distillation residue of | 1020 | 3.7 | 65 |
| oleyl amine (m.p. 25° C.) | | | |
| Distillation residue of oleyl | 950 | 1.3 | 60 |
| amine (m.p. 25° C.) plus 5% | | | |
| (w/w) of tallow fatty amine | | | |
| Distillation residue of | 950 | 9.2 | 110 |
| oleic acid (m.p. 10° C.) | | | |
| Distillation residue of oleic | 1000 | 1.4 | 75 |
| acid (m.p. 10° C.) plus 5% | | | |
| (w/w) of tallow fatty amine | | | |
| Poly(oleic acid) | 950 | 6.5 | 35 |
| Poly(oleic acid) plus 5% | 980 | 1.2 | 60 |
| (w/w) of tallow fatty amine | | | |

This experiment shows that several distillation residues and synthetized polymers of natural origin can be suitable as a main component for fertilizer coatings, as well as for reduction of the baking tendency and dust formation.

### Example 3

For a possibly stronger reduction of the caking of a salt granulate, to fatty acid ester residue 1, 5% (w/w) of polymer on the basis of maleic acid, citraconic acid or itaconic acid, was added. The polymers were prepared by heating the monomer to 180° C. in the presence of an initiator, for example concentrated sulfuric acid. In the same way, mixtures of 20% (w/w) of tallow fatty amine and 80% (w/w) of monomer were prepared. The obtained mixtures were coated onto calcium ammonium nitrate CAN (27% N).

TABLE 6

| | | Baking tendency |
|---|---|---|
| Coating | Dosage (ppm) | (35° C.) (kg) |
| Blank (without a coating) | — | 15 |
| Reference (95% (w/w) of | 850 | 0.6 |
| mineral oil and 5% (w/w) of | | |
| tallow fatty amine) | | |
| Fatty acid ester residue 1 plus 5% | 900 | 3.4 |
| (w/w) of poly maleic acid | | |
| Fatty acid ester residue 1 plus 5% | 860 | 1.2 |
| (w/w) of poly itaconic acid | | |
| Fatty acid ester residue 1 plus 5% | 880 | 2.5 |
| (w/w) of poly citraconic acid | | |
| Fatty acid ester residue 1 plus 5% | 880 | 1.4 |
| (w/w) of a polymer of maleic acid | | |
| and tallow fatty amine | | |
| Fatty acid ester residue 1 plus 5% | 860 | 0.4 |
| (w/w) of a polymer of itaconic | | |
| acid and tallow fatty amine | | |
| Fatty acid ester residue 1 plus 5% | 890 | 0.8 |
| (w/w) of poly citraconic | | |
| acid/tallow fatty amine | | |

The above example shows that polymers on the basis of maleic acid, itaconic acid or citraconic acid can provide a good contribution to the reduction of the baking tendency of salts. The performance of common coating products can be realized when the polymers are mixed with a small amount of tallow fatty amine.

### Example 4

Several salts, among which fertilizers, can be sensitive to moisture absorption. Excessive moisture absorption by salts can result in undesired phenomena such as dust formation and caking. Fatty acid ester residues are, by the presence of the polar ester functionalities, be more hydrophilic than mineral oil and paraffins. To reduce the moisture absorption by salts, stearyl alcohol, bisethoxylated stearyl alcohol or 2-ethyl hexyl phosphate ester has been added to the formulations with a baking tendency lowering effect.

The formulations in Table 7 consist of 90% (w/w) of fatty acid ester residue 1, 5% (w/w) of tallow fatty amine and 5% (w/w) of moisture-protecting compound. These formulations were applied onto calcium ammonium nitrate (CAN, 27 N).

TABLE 7

| | | Moisture |
|---|---|---|
| Active compound | Dosage (ppm) | absorption (g) |
| Blank (without a coating) | — | 11.2 |
| Reference (95% (w/w) of | 1000 | 10.9 |
| mineral oil and 5% (w/w) of | | |
| tallow fatty amine) | | |
| Fatty acid ester residue 1 plus | 950 | 11.0 |
| 5% (w/w) of tallow fatty amine | | |
| Fatty acid ester residue 1, tallow | 1010 | 8.0 |
| fatty amine and stearyl alcohol | | |
| Fatty acid ester residue 1, | 1000 | 8.5 |
| tallow fatty amine and | | |
| bisethoxylated stearyl alcohol | | |
| Fatty acid ester residue 1, | 930 | 8.9 |
| tallow fatty amine and 2-ethyl | | |
| hexyl phosphate ester | | |

The above experiment shows that the application of coatings on the basis of fatty acid ester residue, which are provided with fatty alcohol, whether or not ethoxylated, or an alkyl phosphate ester results in a substantial decrease of the moisture absorption of the treated fertilizer.

### Example 5

In a further experiment it was checked whether fatty alcohol distillation residues can protect fertilizers against moisture absorption. Several products were brought onto magnesium-stabilized calcium ammonium nitrate and checked on performance. As a reference, a typical anti-moisture coating on the basis of n-paraffin, tallow fatty amine and 2-ethyl hexyl phosphate ester was taken. The results are represented in Table 8.

TABLE 8

| | Dosage | Moisture |
|---|---|---|
| Active compound | (ppm) | absorption (g) |
| Blank (without a coating) | — | 11.3 |
| 100% (w/w) of n-paraffin | 620 | 7.9 |
| (m.p. = 45° C.) | | |
| 85% (w/w) of n-paraffin (m.p. = | 580 | 6.7 |
| 45° C.), 7.5% (w/w) of tallow fatty | | |
| amine and 7.5% (w/w) 2-ethyl | | |
| hexyl phosphate ester | | |
| 100% (w/w) of fatty alcohol | 590 | 6.5 |
| distillation residue (C8-C18, | | |
| m.p. = 38° C.) | | |
| 100% (w/w) of fatty alcohol | 600 | 7.1 |
| distillation residue | | |
| (C8-C22, m.p. = 45° C.) | | |
| 80% (w/w) of fatty alcohol | 600 | 8.3 |
| distillation residue (C8-C18), | | |
| m.p. = 38° C.), 20% of | | |
| n-paraffin (m.p. = 45° C.) | | |

The above experiment shows that distillation residues of fatty alcohols are extremely suitable as a anti-moisture coating for extremely moisture-sensible fertilizers such as stabilized nitrate.

### Example 6

To investigate the suitability of polymer distillation residues as a carrier material/solvent for coloring pigments, the following experiment has been executed. Pigment pastes were made by dispersing (Heidolph DIAX 600, 9500-rpm, 5 minutes) 10% (w/w) of blue pigment (Sunfast Blue 15,3) in 80% (w/w) of solvent and 10% (w/w) of stabilizer (PEG-3 tallow fatty amine propylamine). The obtained suspensions were checked for stability and coloring properties on fertilizers.

The polymeric distillation residue 3 shows the same stabilizing properties as those of a low-viscous mineral oil. Apart from that, the distribution on a fertilizer is, good and the color remains maintained for a longer period.

By that it is shown that polymeric distillation residues are suitable as a (co)solvent or carrier material for pigments.

### Example 7

Polymeric distillation residues from biodiesel and related products have also been tested as a density controlling additive for cokes. For that, 1 kg of cokes granulate is treated according to the coating drum method, with 2.0 g of polymeric distillation residues 1, 2 and 3. As a reference, cokes has also been provided with the same dosage of mineral oil. The bulk densities of all treated cokes products are in correspondence with the mineral oil-treated cokes.

This test shows that polymeric distillation residues can act as a density controlling additive for cokes.

### Example 8

Finally, polymeric distillation residues were examined for a defoaming action. For that, a solution of 50 ppm sodium dodecyl benzene sulphonate was prepared. 10 ml solution was transferred in a glass column, being provided at the bottom with a glass filter, a stop cock and an air pump. Before air is bubbled through, 5 ppm of polymer oil (provided or not with 0.25 ppm of silicon oil) is added. After 10 seconds the foam height is determined. As a reference mineral oil is used.

In all circumstances the defoaming action of the distillation residues was observed. The effect can be improved by the addition of silicon oil.

1. A coating composition for the reduction of the tendency to baking, the moisture absorption and the dust formation of a granulate being susceptible therefore, selected from the group consisting of a plant auxiliary substance, fertilizers, minerals, and ores, comprising a polymer compound having formula
((FA)ₚ−X))q (I)
wherein
FA is a saturated or non-saturated, C₈-C₂₄ fatty alkyl, having from 0 to 5 C═C groups in the chain,
X is a residue, selected from the group consisting of —C(O)OH, —C(O)OR, —NH₂, —NHC(O)R′, NHC(O)OH, —OH, and —COR;
p=1 to 1000,
q=1 to 1000,
with the provision that p and q are not both at the same time equal to 1,
R is a lower alkylgroup, and
R′ is H or a lower alkylgroup.

2. A coating composition according to claim 1, wherein R and R′ are each selected from the group consisting of CH₃, C₂H₅, C₃H₇, C₄H₉, C₅H₁₁, pentaerytritol residue, glycerol residue, and an ether bond.

3. A coating composition according to claim 1, wherein FA is a saturated or non-saturated, C₁₄-C₂₂ fatty alkyl, having from 0 to 5 C═C groups in the chain.

4. A coating composition according to claim 1, wherein the polymer compound having formula (I) is derived from the bottom fraction of a biodiesel distillation process.

5. A coating composition according to claim 1, wherein the polymer compound having formula (I) is obtainable by distillation of an ester or salt of a corresponding fatty acid, fatty alcohol, fatty amine, fatty amide, or fatty carbamate having 8-24 C-atoms.

6. A coating composition according to claim 1, wherein the polymer compound having formula (I) is obtainable by polymerization of the corresponding monomer.

7. A coating composition according to claim 6, wherein the polymer compound having formula (I) is obtainable by radical initiated polymerization of the corresponding monomer.

8. A coating composition according to claim 1, wherein the granulate is a fertilizer.

9. A coating composition according to claim 1, wherein the plant auxiliary substance comprises an ammonium nitrate containing fertilizer.

10. A process for the reduction of the tendency to caking, moisture absorption and/or dust formation of a granulate being susceptible therefore, selected from the group consisting of a plant auxiliary substance, fertilizers, minerals and ores, in that the surface of the granulate is at least partially coated with a coating composition according to claim 1.

11. A process according to claim 10, further comprising applying an organo amine having the general formula R″NH₂, wherein R″ is selected from the group consisting of a straight, branched, cyclic and aromatic, C₁-C₃₀ alkyl or alkenyl group.

12. A process according to claim 11, wherein R″ is a straight or branched, C₁₂-C₃₀ alkyl or alkenyl group.

13. A process according to claim 10, further comprising applying onto the granulate at least a compound selected from the group consisting of straight or branched chain, C₁₆-C₂₄ alkane acids; straight or branched chain, C₁₆-C₂₄ alkoxylates; phosphate acid mono en dialkyl esters having 1-24 C-atoms; and straight or branched chain, C₈-C₂₄ alcohols.

14. A process according to claim 10, further comprising applying onto the granulate a polymerization product from a 1,4 unsaturated, C₄-C₁₂ carboxylic acid or carboxylic acid anhydride.

15. A process according to claim 14, wherein said 1,4 unsaturated carboxylic acid or carboxylic acid anhydride, is selected from the group consisting of maleic acid, maleic acid anhydride, itaconic acid, and citraconic acid.

16. A process according to claim 10, further comprising applying onto the granulate paraffin or petrolatum.

17. A process according to claim 10, further comprising applying onto the granulate a surface-active compound.

18. A process according to claim 16, wherein the surface-active compound is an organo silicon containing compound.

19. Use of a composition according to claim 1, for the stabilization of a coloring dispersion comprising coloring pigment particles and a carrier suitable therefore.

20. Use of a composition according to claim 1, for the regulation of the density of cokes particles being obtained according to a common process.

21. A process according to claim 10, wherein the granulate particles are provided with the compound of claim 7, in a dosage of from 100-10,000 ppm, based on the weight of the granulate particles.

22. A process for the preparation of polymer compounds having a formula (I),
((FA)ₚ−X))_{q} (I)
wherein
FA is a saturated or non-saturated, C₈-C₂₄ fatty alkyl, having from 0 to 5 C═C groups in the chain,
X is a residue, selected from the group consisting of —C(O)OH, —C(O)OR, —NH₂, —NHC(O)R′, NHC(O)OH, —OH, and —COR;
p=1 to 1000,
q=1 to 1000,
with the provision that p and q are not both at the same time equal to 1,
R is a lower alkylgroup, and
R′ is H or a lower alkylgroup
wherein a residue flow, which contains vegetable oils, animal fat, or recycled fat, is subjected according to a process known for the production of biodiesel, to transesterification, the thus obtained product is distilled to remove a fraction being suitable as biodiesel, and the thus obtained remaining bottom fraction is isolated to obtain a fraction containing polymer compounds having formula (I), which compounds are, if desired, subjected to functionalization to amine, amide or carbamate compounds.

23. A process according to claim 22, wherein a residue flow known as yellow grease is used.

24. A coating composition according to claim 1, wherein FA is a saturated or non-saturated, C₈-C₂₄ fatty alkyl, having from 0 to 3 C═C groups in the chain.

25. A coating composition according to claim 1, wherein p=1 to 3.

26. A coating composition according to claim 1, wherein q=2 to 5.

27. A coating composition according to claim 3, wherein FA is a saturated or non-saturated, C₁₄-C₂₂ fatty alkyl, having from 0 to 3 C═C groups in the chain.

28. A process according to claim 10, wherein the granulate particles are provided with the compound of claim 7, in a dosage of from 500-3,000 ppm, based on the weight of the granulate particles.
